(19) United States
(12) Patent Application Publication
(10) Pub. No.: US 2016/0257470 A1
(43) Pub. Date: Sep. 8, 2016

(54) APPLICATOR PACKAGE

(71) Applicants: William J. Casey, Whitefish Bay, WI (US); John P. Rowe, Antioch, IL (US)

(72) Inventors: William J. Casey, Whitefish Bay, WI (US); John P. Rowe, Antioch, IL (US)

(21) Appl. No.: 15/058,684

(22) Filed: Mar. 2, 2016

Related U.S. Application Data
(60) Provisional application No. 62/127,158, filed on Mar. 2, 2015.

(51) Int. Cl. B65D 75/30; A45D 40/26; A45D 33/36; A61F 13/40; B65D 75/58; A45D 34/04

(52) U.S. Cl. CPC B65D 75/30; B65D 75/5805; A45D 34/042; A45D 33/36; A61M 35/006; A45D 40/262

(57) ABSTRACT

An applicator package may include a pouch formed from one or more pliable membranes, the pouch comprising a first compartment and a second compartment, the first compartment being separated from the second chamber by a sealing juncture, a material sealed within the first compartment and an applicator having an applicator end in a handle end sealed within the second compartment. The membrane is formed from a material and has a thickness so as to allow the membrane to be manually punctured by the applicator end of the applicator for insertion of the applicator end into contact with the material in the first compartment.

### CROSS REFERENCE TO RELATED APPLICATIONS

The present application claims priority under 35 USC Section 119 from co-pending U.S. Provisional Application Ser. No. 62/127.158 filed on Mar. 2, 2015 by Casey et al. and entitled APPLICATOR PACKAGE, the full disclosure of which is hereby incorporated by reference.

### BACKGROUND

Applicators are sometimes used to apply materials, whether in fluid forms, such as liquid, gels, crèmes or ointments or solid forms. For convenience, the applicators are sometimes packaged into containers with the materials that they contain. Packaging applicators into containers with the materials may lead to a handle of the applicator becoming soiled with the material or may reduce the shelf life of the material within the package.

### BRIEF DESCRIPTION OF THE DRAWINGS

FIG. 1 is a perspective view of an example applicator package.

FIG. 2 is an exploded perspective view of a portion of the example applicator package of FIG. 1.

FIG. 3 is a side view of the portion of the applicator package of FIG. 2.

FIG. 4 is an end view of the portion of the applicator package of FIG. 2.

FIG. 5 is a top view of the portion of the applicator package of FIG. 2.

FIG. 6 is a perspective view of an example interface of the portion of the applicator package of FIG. 2.

FIG. 7 is a side view of the example interface of the portion of FIG. 6.

FIG. 8 is an end view of the example interface of the portion of FIG. 6.

FIG. 9 is a top view of the example interface of the portion of FIG. 6.

FIG. 10 is a perspective view of another example applicator package.

FIGS. 11-13 are top perspective views illustrating one example assembly of the applicator package of FIG. 10.

FIG. 14 is a perspective view illustrating use of the applicator package of FIG. 10.

FIG. 15 is a top view of another example applicator package.

FIG. 16 is a side view of the applicator package of FIG. 15.

FIG. 17 is a side view of a portion of the applicator package of FIG. 15.

FIG. 17A is an end view of the portion of FIG. 17.

FIG. 18 is a fragmentary sectional view of the applicator package of FIG. 10.

FIG. 19 is a top view of a portion of the applicator package of FIG. 10 prior to completion.

FIG. 20 is a side view illustrating assembly of the applicator package of FIG. 10 using the portion of FIG. 19.

FIG. 21 is a top view illustrating use of the applicator package of FIG. 10.

FIG. 22 is a top perspective view of another example applicator package.

FIG. 23 is a sectional view of the example applicator package of FIG. 22.

FIG. 24 is a perspective view of the applicator package of FIG. 22 being used.

FIG. 25 is a perspective view illustrating further use of the applicator package of FIG. 22.

FIG. 26 is a perspective view illustrating further use of the applicator package of FIG. 22.

### DETAILED DESCRIPTION OF THE EXAMPLE EMBODIMENTS

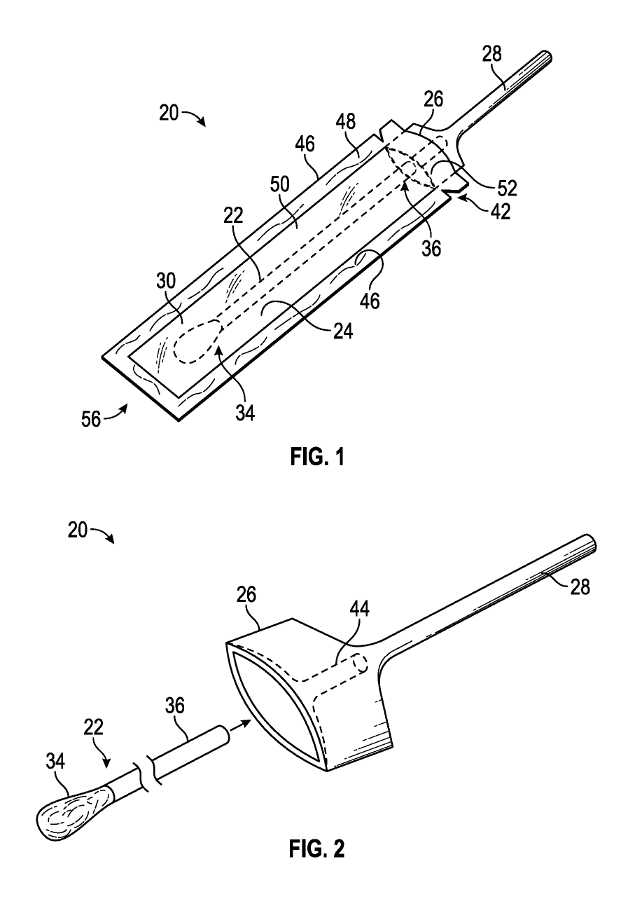

FIG. 1 is a perspective view of applicator package 20 according to one example embodiment. Applicator package 20 contains a fill chemistry 30. Examples of a fill chemistry include, but are not limited to, a fluid, such as a liquid, gels, crèmes or ointments or a solid, such as a powder, granules, or particulates. Applicator package 20 is configured to simultaneously provide the material or fill chemistry and an applicator for applying the material. Package 20 provides a person with a handle for gripping the applicator that is less likely to be soiled, coated or covered with a fill chemistry upon initial use. At the same time, package 20 provides an economical fill chemistry container that has relatively low fabrication and assembly costs and that provides a fill chemistry with an enhanced shelf life.

Applicator package 20 generally includes applicator 22, pouch 24, interface 26 handle 28 and fill chemistry or fill material 30. Applicator 22 comprises a member configured to apply material 30. In the particular example illustrated, applicator 22 includes an applicator end 34 and a handle end 36. Applicator end 34 is configured to hold or carry material 30 as material 30s being applied. In one embodiment, applicator end 34 is absorbent. For example, in one embodiment, applicator end 34 may comprise a swab, such as a cotton swab, a foam, sponge or absorbing fabric or weave. In yet another embodiment, applicator end 34 may comprise a brush, such as a mascara brush. In yet other embodiments, applicator end 34 may be a solid stick such as a spatula, fork, spoon or ball shaped material applying shape. In other embodiments, applicator end 34 may have other configurations.

Handle end 36 extends at an opposite end of applicator 22 proximate to handle 28. In the example embodiment illustrated, applicator end 34 is joined to interface 26 which is joined to handle 28. In other embodiments, handle end 36 may itself serve as a handle to be manually grasped by in individual or person. In such embodiments, handle 28 may be omitted. Although handle end 36 is illustrated as being generally tubular or cylindrical in shape, in other embodiments, handle end 36 may have other configurations. Although handle end 34 is illustrated as being attached to handle 28, in other embodiments, handle end may alternatively be integrally formed as a single unitary body with handle 28.

Pouch 24 comprises a bag, sack or similar receptacle formed from one or more pliable or flexible sheets or membranes. In the particular embodiments illustrated, pouch 24 is formed from two flexible sheets or membranes joined to one another along opposite mutually facing edge surfaces. In other embodiments, pouch 24 may be formed from a single membrane or sheet folded and joined to itself to form the pouch. Because pouch 24 is formed from the flexible sheets or membranes rather than relatively rigid plastic, pouch 24 is lighter in weight, is less expensive and has reduced permeability or breathability, prolonging shelf life of the contents of pouch 24.

In the example embodiment illustrated, pouch 24 is formed from one or more sheets, each sheet having a thickness of about 0.003 inches. In other embodiments, one of a variety of commercial grades of lamenated sheets that are available with some variation in thickness may be used. In one embodiment, each sheet is formed from multiple laminated layers of different materials. In one embodiment, each sheet is formed from a metallic layer, such as aluminum, sandwiched between two polymeric layers, such as a polyester layer and a polyethylene layer. When pouch 24 is formed, the polyethylene layer is on the inside while the polyester layer is on an outside of pouch 24. In other embodiments, pouch 24 may be formed from other polymeric materials, from other metallic materials, and may comprise a fewer or greater number of such layers.

As shown by FIG. 1, pouch 24 additionally includes a sever portion 42 generally between interface 26 and applicator end 34. Sever portion 42 facilitates severing of a portion of pouch 24 from interface 26 so as to permit applicator end 34 to be withdrawn from an interior of pouch 24. In the particular example illustrated, sever portion 42 is configured to be manually torn or ripped to sever pouch 24 from interface 26. In other embodiments, sever portion 42 may be configured to be cut or otherwise broken. In the particular example illustrated, sever portion 42 includes a pair of “tear open” notches. In other embodiments, sever portion 42 may comprise other structures or material treatments configured to facilitate tearing or severing. For example, in other embodiments, sever portion 42 may comprise a groove, channel or partial slit which weakens material along a line to facilitate tearing.

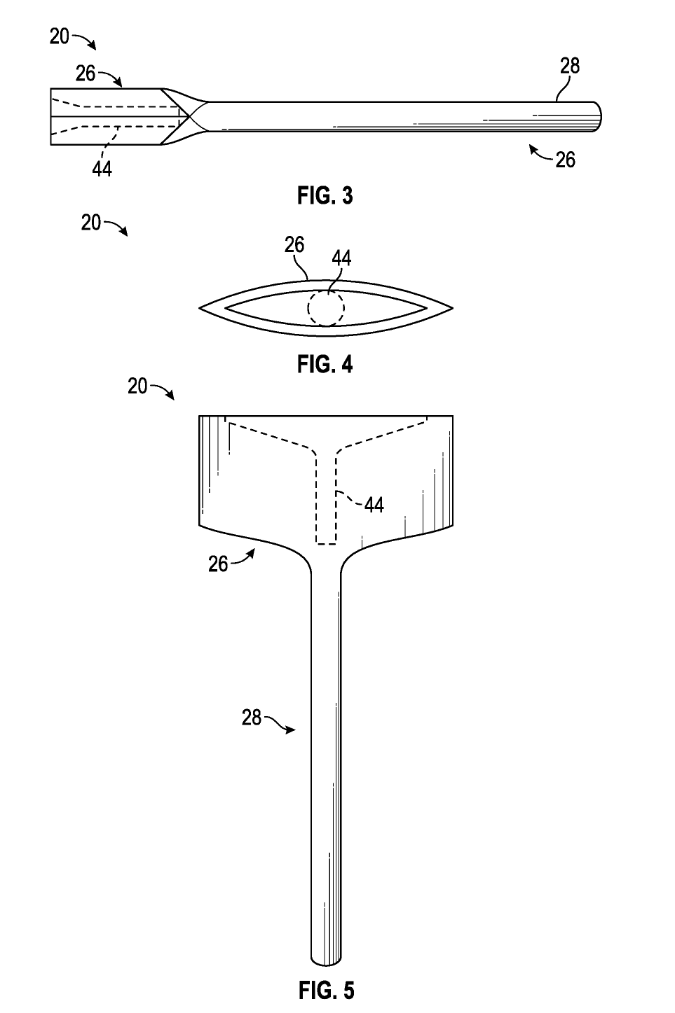

Interface 26 comprises a member having one or more surfaces configured to be sealed to pouch 24. At the same time, interface 26 is configured to be connected or joined to applicator 22 with applicator end 34 located within an interior of pouch 24. FIGS. 2-5 illustrates interface 26 (and handle 28) in more detail. In the particular example illustrated, interface 26 is integrally formed as a single unitary body with handle 28. In other embodiments come interface 26 may be separately formed and joined to handle 28.

As shown by FIG. 4, interface 26 has an irregular cross-sectional shape configured to facilitate sealing of interface 26 to pouch 24. In particular, interface 24 has a flattened shape such that interface 26 is greater in a first dimension and is thinner in a second dimension 90 degrees with respect to the first dimension. In the particular example illustrated, interface 26 has an oval or eye-shaped cross-section. This shape better conforms to the flattened opening of pouch 24. In other embodiments, interface 26 may have other configurations.

As further shown by FIGS. 2, 3 and 5, interface 26 has an internal cavity 44 configured to receive handle end 36 of applicator 22. In the particular about illustrated, cavity 44 is configured to receive handle end 36 such that handle end 36 is press fit and is retained within cavity 44 to secure applicator 22 to interface 26 and handle 28. As a result, applicator 22 may be easily assembled to interface 26. In other embodiments, additional glues, adhesive, welds and the like may be used to further secure handle end 36 to interface 26. In yet other embodiments, cavity 44 may be omitted where other means are used to secure or connect applicator 22 to interface 26.

Handle 28 extends from interface 26 outside of pouch 24 and provides a surface facilitating manual gripping of interface 26 and applicator 22. Because handle 28 extends outside of the interior, fill materials within pouch 24 are less likely to become deposited upon the surface of handle 28 which is grasped by a person. In the particular example illustrated, handle 28 is an elongated rod or shaft. In other embodiments, handle 28 may include a flattened portion providing a greater surface area for gripping. In still other embodiments, handle 28 may have other configurations. Although handle 28 is illustrated as being integrally formed as part of interface 26 to reduce fabrication complexity and cost, in another embodiment, handle 28 may be joined to interface 26 in other fashions.

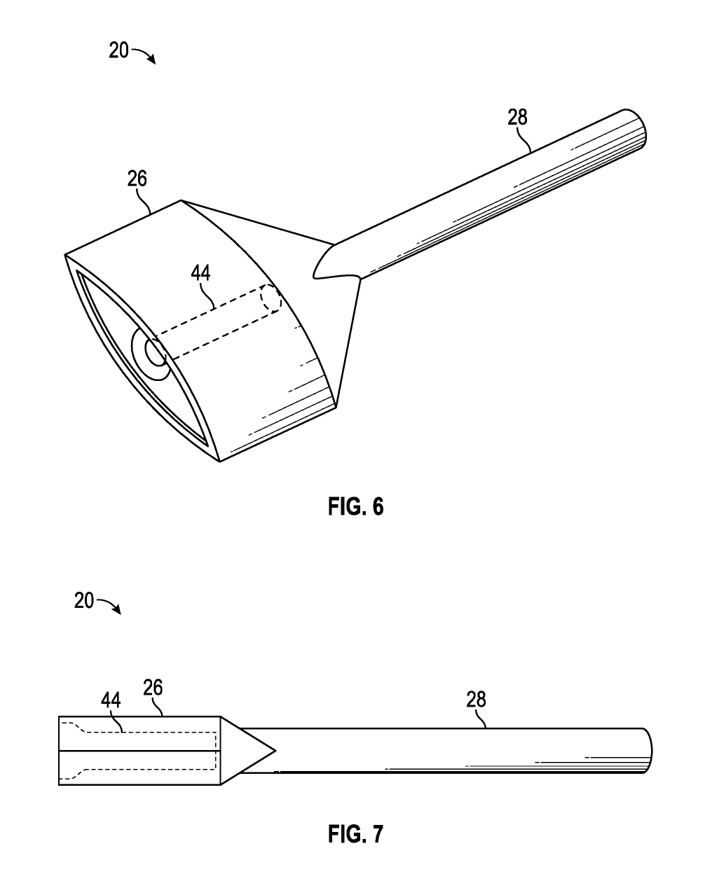

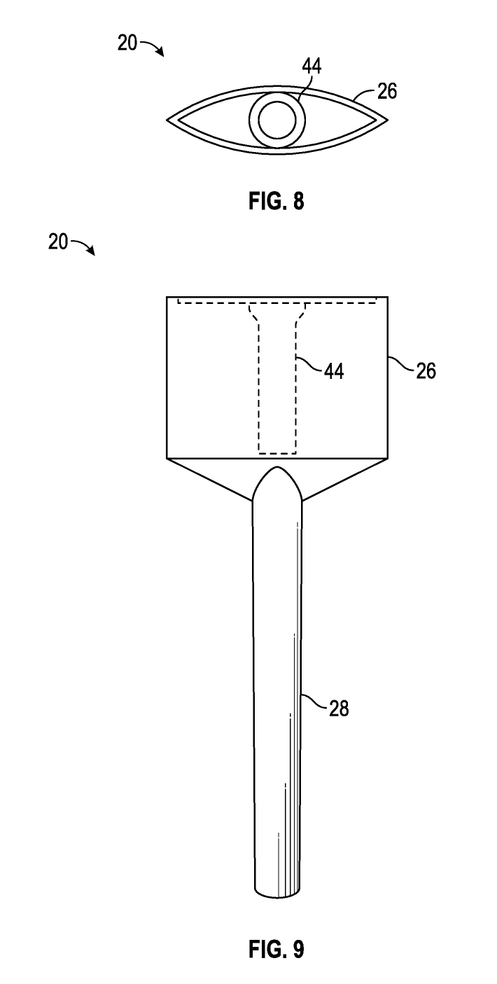

FIGS. 6-9 illustrates one particular embodiments of interface 26 shown in FIGS. 2-5. FIGS. 6-9 correspond to FIGS. 2-5, respectively, and additionally illustrate example dimensions of one example embodiment of interface 26. In other embodiments, interface 26 may have other dimensions as well as other configurations.

According to one embodiment, applicator package 20 may be assembled in the following manner. Applicator end 36 of applicator 22 is inserted into cavity 44 to join applicator 22 to interface 26 and handle 28. Opposite side edges 46 of opposing sheets 48 are heat sealed to one another to form an elongated sleeve having two opposite opened ends. Applicator end 34 is inserted into the interior of the sleeve until the irregular surface of interface 26 is positioned between sheets 48 and partially within the interior 50 of the sleeve. Thereafter, portions 52 of pouch 24 are heat sealed to and about interface 26 to close one end of pouch 24. Solid or fluid material 30 is then inserted into the interior of pouch 24 through the opening at end 56. After a desired amount of material 30 is placed within the interior 50, end 56 of pouch 24 is heat sealed.

In other embodiments, end 56 may be sealed and material 30 may be inserted into interior 50 prior to applicator and 30 for being inserted into interior 50 and prior to portions 52 of pouch 24 being heat sealed or otherwise sealed to interface 26.

In still other embodiments, other assembly steps may be employed. For example, in another embodiment, a single sheet may be used, wherein pouch 24 having one closed end is first formed by folding a single sheet and sealing opposite surfaces to one another such that pouch 24 has a single open end. After material is inserted into the interior of the pouch, interface 26 and applicator end 34 are inserted into interior 50 and sealed to pouch 24.

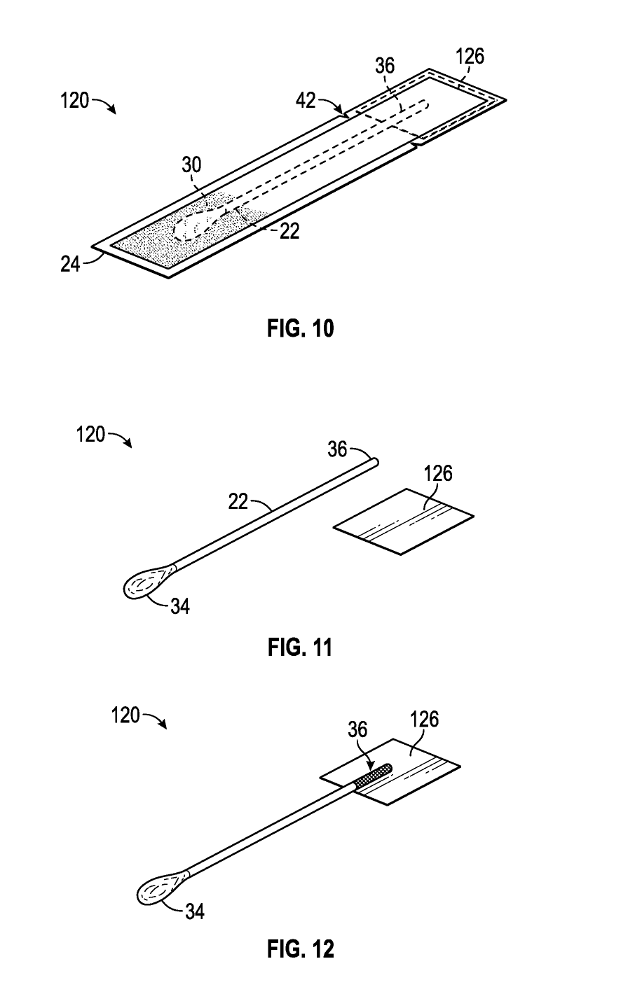

FIGS. 11-14 illustrate applicator package 120, another embodiment of applicator package 20. FIG. 10 illustrates a completed applicator package 120. Applicator package 120 includes applicator 22, pouch 24 and interface 126. Applicator 22 and pouch 24 are substantially similar to applicator 22 illustrated and described above with respect to applicator package 20 except that applicator 22 and pouch 24 are alternatively secured to interface 126 in lieu of interface 26.

Interface 126 comprises a structure joined to applicator 22 and also joined to pouch 24. Interface 126 facilitates coupling of applicator 22 to pouch 24. In the example illustrated, applicator 22 is joined to interface 126 at a first location while pouch 24 is joined to interface 126 at a second distinct location or sets of locations spaced from the first location. In other words, interface 126 is not joined to both applicator 22 and pouch 24 at a common location. As a result, Interface 126 facilitates coupling of applicator 22 to pouch 24 while at least substantially maintaining the integrity of the seal provided by pouch 24 about the contents of pouch 24.

In the particular example embodiment shown, interface 126 comprises a tab or other flat sheet or panel of material. In one embodiment, interface 126 comprises a tab having an outer surface formed from the same general material as the inner surface of pouch 24 to enhance welding or fusing of interface 126 to pouch 24. For example, in one embodiment, the interior surface of pouch 24 is formed from polyethylene while one or both exterior surfaces of interface 126 are also formed from polyethylene. In still other embodiments, such surfaces may be formed from other adhesives, glues or fusible polymeric materials other than polyethylene. In one embodiment, interface 126 is entirely formed from polyethylene.

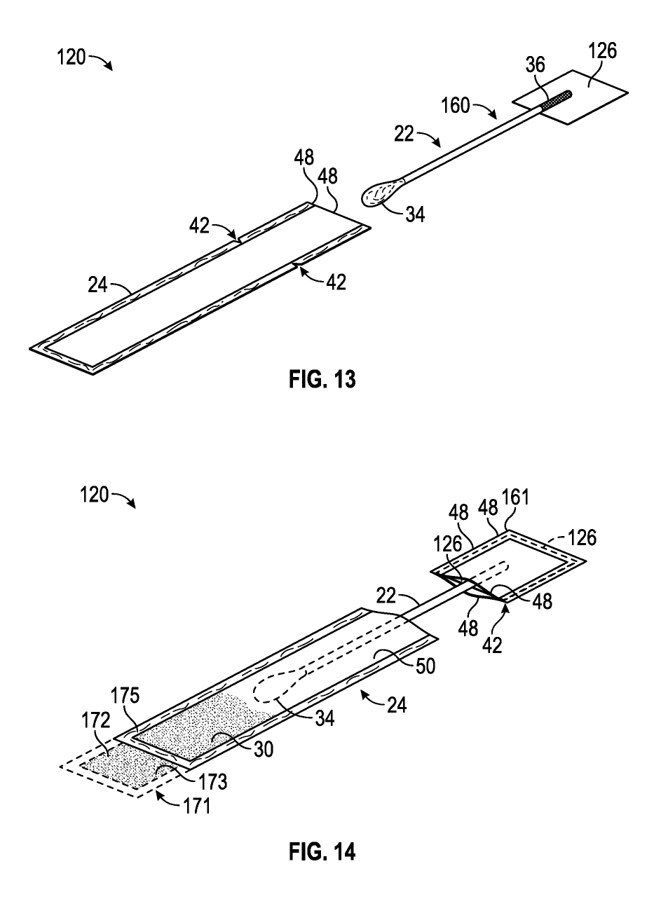

FIGS. 11-13 illustrate assembly of applicator package 120 according to one example embodiment. As shown by FIG. 11, applicator 22 and interface 126 are provided. As shown by FIG. 12, applicator end 36 of applicator 22 is joined to interface 126. In one embodiment, handle end 36 is heat welded to interface 126. In other embodiments, glues, adhesives or other fasteners may be used for such joining As shown by FIG. 13, the applicator interface subassembly 160 is inserted into pouch 24 and pouch 24 is filled with material 30. In one embodiment, subassembly 160 is inserted into pouch 24 and portions of pouch 24 opposite to interface 126 are heat sealed to interface 126 to close off one end of pouch 24. Thereafter, material 30 is inserted into pouch 24 through the other open end and the other open and is subsequently heat sealed or heat welded. In another embodiment, pouch 24 is heat sealed so as to form a closed end and is filled with material 30 through the open end. Thereafter, subassembly 160 is inserted into the interior of the pouch through the open end and portions of pouch 24 opposite to interface 126 are heat sealed to interface 126.

According to one embodiment, both opposing sheets 48 of pouch 24 are heat sealed to opposite faces of interface 126. In other embodiments, only one of such sheets 48 is heat sealed to a single face of interface 126. In the embodiment illustrated, one end of pouch 126 may be close or sealed simultaneous with the joining of pouch 24 to interface 126. In other embodiments, interface 126 may be joined to pouch 24 in other fashions.

FIG. 14 illustrates the use of applicator package 120. As shown by FIG. 14, a person first severs pouch 24 using sever portion 42 of pouch 24. Thereafter, the user may grasp portion 161 of pouch 24 (and portion 161 is firmly affixed to applicator end 36) which serves as a handle. Using portion 161, which has an enlarged more easily grippable surface area, a person may withdraw applicator end 34 from interior 50 of pouch 24 for use. Because outer surfaces of portion 161 are less likely to become exposed or coated with the material 30 within interior 50, a person is less likely to get material 30 upon his or her hand during use.

As shown by broken lines in FIG. 14, in another embodiment, applicator package 120 may additionally include an additional pouch 171 having an interior 172 containing an additional material 173 distinct from or the same as material 30. In such an embodiment, pouch 24 and pouch 171 may be separated from one another by an intermediate seal 175. Seal 175 may be configured to be broken or otherwise opened, permitting material from interior 172 to enter interior 50 for mixing with material 30 and for removal an application by applicator 22. To facilitate communication between the interiors of pouches 171 and 24, a user may simply press or squeeze at least a portion of pouch 24, pouch 171 or the juncture 175 therebetween. Alternatively, this additional pouch 171 may be omitted.

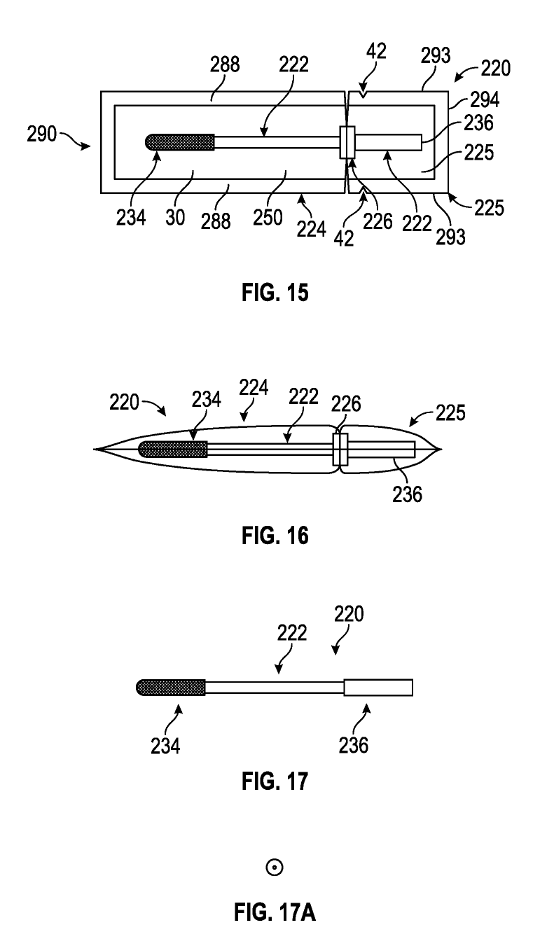

FIGS. 15-21 illustrate applicator package 220, yet another embodiment of applicator package 20. As shown by FIGS. 15-17A applicator package 220 includes applicator 222, pouch 224, pouch 225 and pouch interface 226. As shown by FIG. 17, applicator 222 comprises an elongated member having an applicator end 234 and a handle end 236. Applicator end 234 is configured to hold or carry material 30 as material 30 is being applied. In one embodiment, applicator end 234 may comprise a brush, such as a mascara brush. In another embodiment, applicator end 234 is absorbent. For example, in one embodiment, applicator end 234 may comprise a swab, such as a cotton swab, a foam, sponge or absorbing fabric or weave. In other embodiments, applicator end 234 may have other configurations.

Handle end 236 extends at an opposite end of applicator 222. In the example embodiment illustrated, applicator end 34 has an enlarged dimension, such as an enlarged diameter (shown in FIG. 17A) which is larger than openings of pouch interface 226, inhibiting handle end from passing through interface 226. In the particular example illustrated, handle end 236 is configured to serve as a handle upon withdrawal from pouch 225. In other embodiments, handle end 236 may be joined to pouch 225 within pouch 225, wherein handle end 236 and at least a portion of pouch 225 provide a surface for being gripped or grasped by a person manipulating applicator 222 during use. In such an embodiment, handle end 236 may alternatively have a dimension or diameter smaller than the one or more openings of interface 226. Although handle end 236 is illustrated as having a generally circular cross-sectional shape, in other embodiments, handle end 236 may comprise a flattened plate-like member or other structure providing a surface to be manually gripped. In yet other embodiments, handle end 236 may be configured to be attached to a separate handle upon withdrawal from pouch 225. For example, in another embodiment, handle end 236 may alternatively include external threads or an internal bore having internal threats configured to be screwed to a separate handle component upon withdrawal from pouch 225. In still other embodiments, handle end 236 may include one or more detents or one or more depressions configured to be releasably engaged by one or more projections or one or more detents of a corresponding separate handle component to facilitate snapping or other securement of a separate handle component to handle end 236 upon withdrawal from pouch 225. In such an embodiment, customized or reusable handle components may be provided for use with applicator package 220.

Pouch 224 comprises a container structure extending about applicator end 234 of applicator 222. In one embodiment, pouch 224 is formed from one or more pliable or flexible sheets or membranes. As shown by FIG. 19, in the particular embodiment illustrated, pouch 224 is formed from a single flexible sheet or membrane 248 having an opening 249, wherein sheet 248 is folded such that opposite mutually facing surfaces of the folded sheet may be joined to one another. In other embodiments, pouch 224 may be formed from multiple membranes or sheets joined to one another to form the pouch. Because pouch 224 is formed from a flexible sheet or membrane rather than relatively rigid plastic, pouch 224 is lighter in weight, is less expensive and has reduced permeability or breathability, prolonging shelf life of the contents of pouch 224.

In the example embodiment illustrated, pouch 224 is formed from one sheet having a thickness of about 0.003 inches (thickness may vary based on exact commercial grade of sheet selected). In one embodiment, the sheet is formed from multiple laminated layers of different materials. In one embodiment, the sheet is formed from a metallic layer, such as aluminum, sandwiched between two polymeric layers, such as a polyester layer and a polyethylene layer. When pouch 224 is formed, the polyethylene layer is on the inside' for while the polyester layer is on and outside of pouch 224. In other embodiments, pouch 224 may be formed from other polymeric materials, from other metallic materials, and may comprise a fewer or greater number of such layers. In addition, in other embodiments, applicator package 220 may additionally include another pouch 171 adjacent to pouch 24 and having an interior 173 separated from the interior of pouch 24 by a selectively breakable or openable sealed juncture as described above with respect to applicator package 120.

As shown by FIGS. 15 and 16, pouch 225 comprises a container structure extending about handle end 234 of applicator 222. In one embodiment, pouch 225 is formed from one or more pliable or flexible sheets or membranes. FIG. 19 illustrates sheet 251 from which pouch 225 is formed. FIG. 19 illustrates sheet 251 overlying sheet 248 of pouch 224 to illustrate relative proportions in the example embodiment. As shown by FIG. 19, in the particular embodiment illustrated, pouch 225 is formed from a single flexible sheet or membrane 251 having an opening 253, wherein sheet 251 is folded such that opposite mutually facing surfaces of the folded sheet may be joined to one another. In other embodiments, pouch 225 may be formed from multiple membranes or sheets joined to one another to form the pouch. Because pouch 225 is formed from a pliable flexible sheet or membrane, such as a foil sheet, rather than relatively rigid plastic, pouch 224 is lighter in weight, is less expensive and has reduced permeability or breathability, prolonging shelf life of the contents of package 220.

In the example embodiment illustrated, pouch 225 is formed from one sheet having a thickness of about 0.003 inches (thickness may vary based on exact commercial grade of sheet selected). In one embodiment, the sheet is formed from multiple laminated layers of different materials. In one embodiment, the sheet is formed from a metallic layer, such as aluminum, sandwiched between two polymeric layers, such as a polyester layer and a polyethylene layer. When pouch 225 is formed, the polyethylene layer is on the inside' for while the polyester layer is on and outside of pouch 225. In other embodiments, pouch 225 may be formed from other polymeric materials, from other metallic materials, and may comprise a fewer or greater number of such layers.

As shown by FIGS. 15 and 19, pouch 225 additionally includes a sever portion 42 on an opposite side of the interface 226 as applicator end 34. Sever portion 42 facilitates severing of a portion of pouch 225 from interface 226 so as to permit applicator end 234 to be withdrawn from an interior of pouch 224. In the particular example illustrated, sever portion 42 is configured to be manually torn or ripped to sever pouch 225 from interface 226. In other embodiments, sever portion 42 may be configured to be cut or otherwise broken. In the particular example illustrated, sever portion 42 includes a pair of “tear open” notches. In other embodiments, sever portion 42 may comprise other structures or material treatments configured to facilitate tearing or severing. For example, in other embodiments , sever portion 42 may comprise a groove, channel or partial slit which weakens material along a line to facilitate tearing.

Pouch interface 226 joins and seals between pouches 224 and 225 while permitting applicator end 234 to be positioned within interior 250 of pouch 224 and permitting handle end 236 to be positioned within interior 255 of pouch 225. As a result, the overall structure, prior to severing sever portion 42, provides a continuous seal between pouches 224, 225 and interface 226. In the particular example embodiment illustrated, interface 226 further wipes applicator end 234 as applicator 222 is being withdrawn through interface 226.

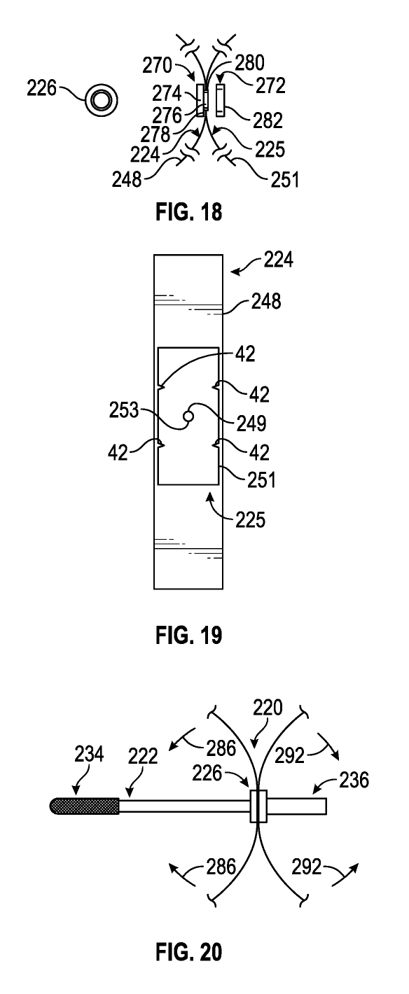

As shown by FIGS. 17 and 18, interface 226 comprises members 270 and 272. Member 270 includes an a first opening 274, a second opening 276, a shoulder surface 278 end hub 280 Opening 274 has a dimension larger than at incompressible supporting portion of applicator end 234 yet smaller than an outermost dimension of the compressible portion of applicator end 234. As a result, during withdrawal of applicator end 234 through opening toward 74, member 270 then, deforms, compresses or otherwise wipes applicator end 234 to remove excess material from applicator end 234. In particular, an entire outer circumference of applicator end 234 it's concurrently compressed, bent or wiped during withdrawal of applicator 222. In other embodiments, opening 274 may have a dimension greater than the outermost dimension of applicator end 234. In the example embodiment illustrated, member 270 comprises a retainer nut. In other embodiments, member 270 may have other configurations.

Opening 276 has a dimension generally greater than an outer dimension of applicator end 234 but less than an outer dimension of the handle end 236. As a result, opening 276 inhibits passage of handle end 236 through interface 226. In other embodiments, opening 276 may have a diameter or interior dimension greater than the outer diameter or dimension of handle end 236.

Shoulder surface 278 extends around hub 280. Shoulder surface 278 cooperates with member 272 to capture sheets 248 and 251 of pouches 224 and 225 there between. In one embodiment, shoulder surface 278 is sealed to sheet 248 while member 272 is sealed to sheet 251, such as by heat welding. In other embodiments, shoulder surface 278 may be omitted.

Hub 280 projects from shoulder surface 278 and is configured to pass through member 272. Hub 280 has an outer circumferential surface sealed to an inner circumferential surface of member 272. In other embodiments, the outer facing surface of hub 280 or an axial facing surface of hub 280 many alternatively be sealed to sheet 251 and/or sheet 248. In one embodiment, hub 280 may be provided with an axial face formed from a compressible sealing material, such as rubber or the like, such that the axial face seals against handle end 236 to provide an additional barrier to contents within pouch 244. In another embodiment, those portions of handle end 236 which about the axial face or end of pub 280 may be formed from one or more compressible sealing materials, such as rubber and the like, to form a gasket for providing an additional barrier to the contents of pouch 224. In yet other embodiments, this feature may be omitted.

Member 272 comprises a member having an opening 280. Opening 280 receives hub 280 while member 272 about shoulder surface 278. As noted above, inner circumferential surface of opening 282 is sealed against an outer circumferential surface of hub 280. As a result, members 270 and 272, being sealed to one another, joining pouches 224 and 225 while forming a sealed interlock therebetween. In the particular embodiment illustrated, member 272 comprises a washer. In other embodiments, members 270 and 272 may have other configurations.

FIGS. 17 and 20 illustrate assembly of applicator package 220. As shown by FIG. 17, openings 249 and 253 of sheets 248 and 251, respectively, are aligned and placed about hub 280 of member 270. Member 272 is also positioned about hub 280 capturing sheets 248 and 251 against shoulder surface 278. At some point in time, portions of sheet 248 about opening 249 and portions of sheet 251 about opening 253 are sealed to at least one of shoulder surface 278, hub 280 or member 272. Member 272 is further sealed to at least one of member 270 and sheet 251. In the example illustrated, sheet 248 is heat sealed or heat welded to shoulder surface 278. Sheet 253 is heat sealed or heat welded to an axial face of member 272. An inner circumferential surface of opening 282 is heat sealed or heat welded to an outer circumferential surface of hub 282, completing the sealed juncture. As shown by FIG. 20 and indicated by arrows 286 sheet 248 is folded away from interface 226 and it's opposite side edges 288 (shown in FIG. 15) are sealed to one another, leaving end 290 open. As indicated by arrows 292 (shown in FIG. 20) sheet 251 is folded away from interface 226 about handle end 236. As shown by FIG. 15, opposite side edges 293 and end surfaces 294 are sealed to one another, such as by heat welding or fusing. Consequently, pouch 225 is closed but for any opening through interface 226. Material 30, a solid or a fluid, is subsequently placed into interior 250 through open end 290 (shown in FIG. 15). Once an adequate or desired amount of material 30 has been placed within interior 250, end 290 is also sealed or closed such as by heat or fusing or heat welding. As a result, interiors of both pouches 224 and 225 are closed to the outside. Communication between such pouches may occur through interface 226. However, as noted above, in particular embodiments, at least partial seal may be formed between handle and 236 and interface 226.

During use of package to 20, pouch 225 is severed at sever portion 42. Thereafter, a person may grasp either the remaining portion of pouch 225 about applicator end 236 or may withdraw applicator end 236 from the remainder of pouch 225 to directly grasp of applicator end 236. As noted above, in some embodiments, a person may alternatively secure a separate handle component to applicator end 236.

Applicator 222 may be manually withdrawn from pouch 224 through interface 226. During such withdrawal, interface 226 wipes applicator end 234 to remove at least some of material 30 upon applicator end 234. In other embodiments, such wiping action may be omitted. Upon withdrawal, applicator 222 may be used to apply material 30. For example, in one embodiment in which applicator package 220 contains mascara, applicator 222 may be used to apply mascara. In particular embodiments, after use, applicator package 220 may be disposed.

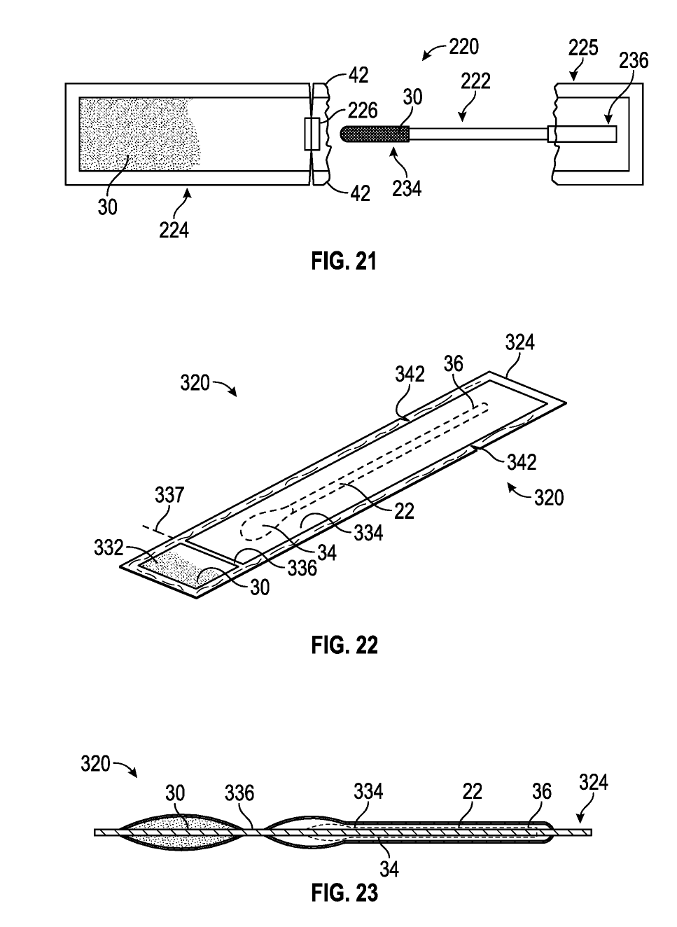

FIGS. 22-23 illustrate applicator package 320. FIG. 22 is a sectional view of the applicator package 320 shown in FIG. 21. Applicator package 320 comprises applicator 22, pouch 324 and fill chemistry or fill material 30. Applicator 22 and fill material 30 are described above.

Pouch 324 comprises a bag, sack or similar receptacle formed from one or more pliable or flexible sheets or membranes. In the particular embodiments illustrated, pouch 324 is formed from two flexible sheets or membranes joined to one another along opposite mutually facing edge surfaces. In other embodiments, pouch 324 may be formed from a single membrane or sheet folded and joined to itself to form the pouch. Because pouch 324 is formed from the flexible sheets or membranes rather than relatively rigid plastic, pouch 324 is lighter in weight, is less expensive and has reduced permeability or breathability, prolonging shelf life of the contents of pouch 324.

In the example embodiment illustrated, pouch 324 is formed from one or more sheets, each sheet having a thickness of about 0.003 inches. In other embodiments, one of a variety of commercial grades of laminated sheets that are available with some variation in thickness may be used. In one embodiment, each sheet is formed from multiple laminated layers of different materials. In one embodiment, each sheet is formed from a metallic layer, such as aluminum, sandwiched between two polymeric layers, such as a polyester layer and a polyethylene layer. When pouch 324 is formed, the polyethylene layer is on the inside while the polyester layer is on an outside of pouch 324. In other embodiments, pouch 324 may be formed from other polymeric materials, from other metallic materials, and may comprise a fewer or greater number of such layers.

Pouch 324 comprises compartments 332 and 334 adjacent one another, but sealed off from one another by an intermediate sealing juncture 336. Compartment 332 contains the fill chemistry or fill material 30 while compartment 334 contains applicator 22. Sealing juncture 336 seals fill material 30 within compartment 332. Sealing juncture 336 comprises a membrane, film, adhesive or fused joint formed from one or more materials that separate compartments 332, 334 to keep the chemistry contained in compartment 332 reasonably separated from applicator 22 in chamber or compartment 334 (although minute/trace amounts of air/chemistry may seep back and forth between such compartments in some implementations), but wherein juncture 336 has a thickness and strength to facilitate puncturing of sealing juncture 336 by applicator end 34 of applicator 22 at the intended time of use. Sealing juncture 336 is formed from one or more materials that have a thickness such that a person may manually grip handle end 36 and push applicator end 34 through and across sealing juncture 336, puncturing sealing juncture 336, to position applicator and 34 into contact with fill material 30.

In one implementation, sealing juncture 336 comprises the same material forming the rest of pouch 324, wherein sealing juncture 336 has a thickness so as to be manually punctured or pulled apart by applicator end 34 while, at the same time, the seal created along the opposite side edges 46 of opposing sheets 48, along a perimeter of pouch 324, will fail or will not be punctured or pulled apart while sealing juncture 336 is punctured or pulled apart or under the same force necessary to puncture or pull apart juncture 336. As a result, the integrity of pouch 324 is maintained even as juncture 336 is opened. In one implementation, sealing juncture 336 comprises a webbing or panel formed between and extending between the two main panels forming opposite faces of pouch 324. In another implementation, sealing juncture 336 is formed by a fused or welded joint between the two main panels forming opposite faces of pouch 324. For example, in one implementation, sealing juncture 336 is formed by applying heat two opposite faces of the two main panels forming part 324 along the axis 337 so as to melt and fuse portions of the two main panels to one another along axis 337 transversely across pouch 324. In yet another implementation, sealing juncture 336 is formed by applying a liquid material or adhesive along axis 337 to one or both of the mutually facing sides of the two main panels forming pouch 324, wherein the liquid material is subsequently dried or cured, forming a solid compartment separating and compartment sealing interface, joint, panel or membrane.

Sealing juncture 336 is configured so as to be manually punctured by applicator end 34. In one implementation, sealing juncture 336 and the perimeter seal forming the outer edges of pouch 324 are very different. In one implementation, pouch 324 is formed as follows. First, the two long edges and one short edge, adjacent compartment 332 are sealed such as by heat fusing such edges to one another or by application of an intermediate adhesive. Second, the fill material 30 is inserted into or through the open and of the partially completed pouch 324, filling the end of pouch 324 adjacent to the sealed short edge. Third, sealing juncture 336 is formed or closed. In one implementation, opposite faces of the two main panels forming pouch 324 are heat fused to one another to form juncture 336. In yet other implementations, a separate membrane is added and joined within the interior of pouch 324 or an adhesive is applied across and between the opposing layers of pouch 324 to form juncture 336. Fourth, applicator 22 is inserted into the dust formed compartment 334. Fifth, the remaining open short edge of pouch 324 is sealed. Such sequential sealing and formation of the outer perimeter edges of pouch 324 and juncture 336 facilitates provision of much stronger robust perimeter edges and a much weaker juncture 336 capable of being punctured by applicator 22. In one implementation, juncture 336 is formed or configured such that a user/consumer may push applicator 22 through juncture 336 using force of 5 pounds or less and nominally between one and 3 pounds of force. The strength of the seal along the outer edges 46 is much greater than the strength of juncture 336. In other implementations, sealing juncture 336 may have other configurations.

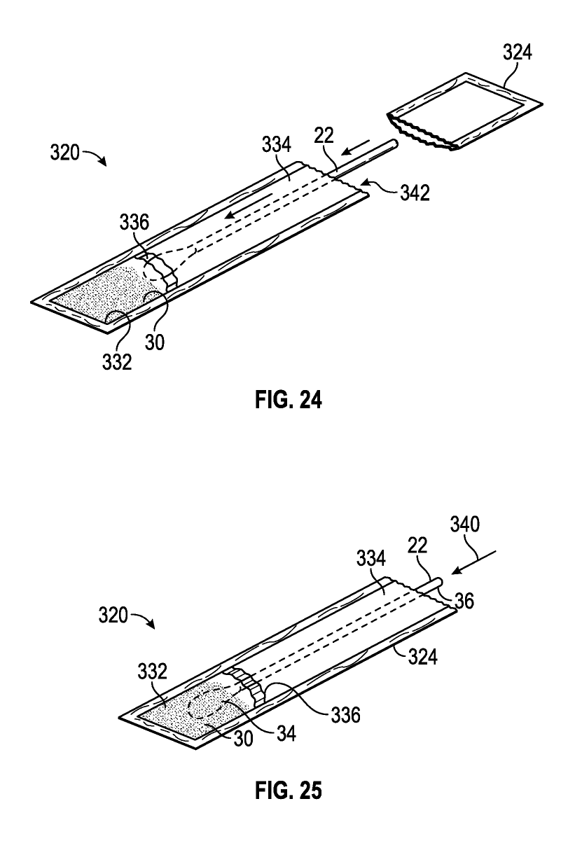

As shown by FIG. 24, pouch 324 additionally includes a break or sever portion 342 adjacent applicator end 34 of applicator 22. Sever portion 342 facilitates severing of a portion of pouch 24 so as to permit applicator 22 to be withdrawn from an interior of pouch 324. In the particular example illustrated, sever portion 342 is configured to be manually torn or ripped to sever pouch 324. In other embodiments, sever portion 342 may be configured to be cut or otherwise broken. In the particular example illustrated, sever portion 342 includes a pair of “tear open” notches. In other embodiments, sever portion 342 may comprise other structures or material treatments configured to facilitate tearing or severing. For example, in other embodiments, sever portion 342 may comprise a groove, channel or partial slit which weakens material along a line to facilitate tearing. The location of tear sever portion 342 is made along the edge of pouch 324 so that at least ¼″ of handle end 36 of applicator 22 is exposed after pouch 324 is removed to enable the user's fingers to easily grasp and then subsequently manipulate applicator 22.

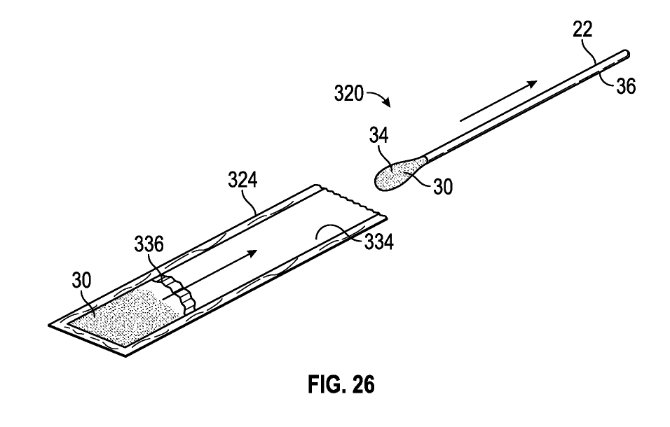

FIGS. 24-26 illustrate use of applicator package 320. As shown by FIG. 24, pouch 324 is severed at sever portion 342. Thereafter, a person may grasp handle end 36. As shown by FIG. 25, applicator 22 may be manually pushed, in the direction indicated by arrow 340, through sealing juncture 336 to move applicator end 34 into a contact with or immersion into the fill material 30. As shown by FIG. 26, applicator 22 may be manually withdrawn from pouch 324. In some implementations, as described above, handle end 36 of applicator 22 is fused, bonded or adhered to an end portion of pouch 324 on an opposite side of sealing junction 336 as chamber 332, wherein the torn away end portion of pouch 324 serves as a handle for applicator 22. Upon withdrawal, applicator 22 may be used to apply material 30. For example, in one embodiment in which applicator package 320 contains mascara, applicator 22 may be used to apply mascara. In particular embodiments, after use, applicator package 320 may be disposed.

In other implementations, juncture 336 is configured to be manually broken in other fashions. For example, in other implementations, juncture 336 is configured to be broken by squeezing rather than the use of and 34 of applicator 22. In one implementation, the geometry/orientation of chambers or compartment 322, 334 is such that the breakage of seal or juncture 336 disperses a chemical fill material at a right angle to the inserted and 34 of applicator 22 (perpendicular to the longitudinal or long/major length or dimension of pouch 324). As a result, the chemistry or fill material 30 is more likely to swab end 34 rather than flowing towards the handle end 36 of applicator 22.

Although the present disclosure has been described with reference to example embodiments, workers skilled in the art will recognize that changes may be made in form and detail without departing from the spirit and scope of the defined subject matter. For example, although different example embodiments may have been described as including one or more features providing one or more benefits, it is contemplated that the described features may be interchanged with one another or alternatively be combined with one another in the described example embodiments or in other alternative embodiments. Because the technology of the present disclosure is relatively complex, not all changes in the technology are foreseeable. The present disclosure described with reference to the example embodiments and set forth in the following claims is manifestly intended to be as broad as possible. For example, unless specifically otherwise noted, the claims reciting a single particular element also encompass a plurality of such particular elements.

What is claimed is:

1. An applicator package comprising:
a pouch formed from one or more pliable membranes, the pouch comprising a first compartment and a second compartment, the first compartment being separated from the second chamber by a sealing juncture;
a material sealed within the first compartment;
an applicator having an applicator end and a handle end sealed within the second compartment, wherein the membrane is formed from a material and has a thickness so as to allow the membrane to be manually punctured by the applicator end of the applicator for insertion of the applicator end into contact with the material in the first compartment.

2. The applicator package of claim 1, wherein the pouch further comprises sever portions to facilitate separation of an end portion of the second compartment to access the handle end of the applicator.

3. The applicator package of claim 1, wherein the one or more pliable membranes comprise at least one foil.

4. The applicator package of claim 1, wherein the one or more pliable membranes includes a transparent membrane.

5. The applicator package of claim 1, wherein the applicator end is configured to hold a fluid.

6. The applicator package of claim 5, wherein the applicator end comprises a swab.

7. The applicator package of claim 1, wherein the material comprises a fluid.

8. The applicator package of claim 1, wherein the pouch comprises:
a first sheet on a first side of the applicator and a second sheet on a second opposite side of the applicator and sealed to at least one of the tab and the first sheet about the handle end, wherein the first sheet and the second sheet are sealed to one another along opposite edges about the applicator and to form a chamber about the applicator end.

9. An applicator package comprising:
an applicator having an applicator end and a handle end;
a pouch formed from one or more pliable membranes extending about the applicator end; and
an interface joined to the handle end and sealed to the pouch.

10. The applicator package of claim 9, wherein the interface is integrally formed as a single unitary body with the handle end.

11. The applicator package of claim 9, wherein the interface has an oval or eye-shaped cross-section at a junction of the interface and the pouch.

12. The applicator package of claim 9, wherein the one or more pliable membranes includes a transparent membrane.

13. The applicator package of claim 9, wherein the pouch is configured to hold a fluid.

14. The applicator package of claim 9, wherein the applicator end comprises a swab.

15. The applicator package of claim 9, wherein the pouch includes a sever portion between the interface and the applicator end.

16. The applicator package of claim 9, wherein the membrane includes a metal layer sandwiched between two polymeric layers.

17. The applicator package of claim 9 further comprising a material sealed within the pouch about the applicator end.

18. The applicator package of claim 9, wherein the pouch comprises:
a first sheet on a first side of the applicator and a second sheet on a second opposite side of the applicator and sealed to at least one of the tab and the first sheet about the handle end, wherein the first sheet and the second sheet are sealed to one another along opposite edges about the applicator and to form a chamber about the applicator end.

19. The applicator package of claim 9, wherein the interface receives the handle end.

20. The applicator package of claim 9, wherein the interface includes a handle portion extending from the handle end.
